(19) United States
(12) Patent Application Publication
(10) Pub. No.: US 2025/0090520 A1
(43) Pub. Date: Mar. 20, 2025

(54) DERIVATIVES OF TURBINMICIN AS ANTIFUNGAL AGENTS

(71) Applicant: Wisconsin Alumni Research Foundation, Madison, WI (US)

(72) Inventors: Timothy Scott Bugni, Madison, WI (US); Weiping Tang, Madison, WI (US); Le Guo, Madison, WI (US); Changgui Zhao, Madison, WI (US); Fan Zhang, Madison, WI (US); Douglas R. Braun, Madison, WI (US); David Andes, Madison, WI (US); Miao Zhao, Madison, WI (US); Jenna Lee Fossen, Madison, WI (US)

(73) Assignee: Wisconsin Alumni Research Foundation, Madison, WI (US)

(21) Appl. No.: 18/695,800

(22) PCT Filed: Sep. 26, 2022
(86) PCT No.: PCT/US2022/044739

§ 371 Date: Mar. 26, 2024

Related U.S. Application Data
(60) Provisional application No. 63/249,490, filed on Sep. 28, 2021.

(51) Int. Cl. A61K 31/4741; A61K 31/4196; A61K 31/429; A61K 31/4439; A61K 31/496; A61K 31/513; A61K 31/5377; A61K 31/7048; A61K 38/12; A61P 31/10; C07D 491/16

(52) U.S. Cl. CPC A61K 31/4741; A61K 31/4196; A61K 31/429; A61K 31/4439; A61K 31/496; A61K 31/513; A61K 31/5377; A61K 31/7048; A61K 38/12; A61P 31/10; C07D 491/16

(57) ABSTRACT

Turbinmicin analogs of Formula I are provided. Compositions including Turbinmicin analogs of Formula I, such as pharmaceutical compositions including effective amounts of Turbinmicin analogs of Formula I for treating fungal infections such as Candida and Aspergillus, including drug-resistant strains thereof, are also disclosed. Methods of treating fungal infections with Turbinmicin analogs of Formula I and compositions thereof are disclosed.

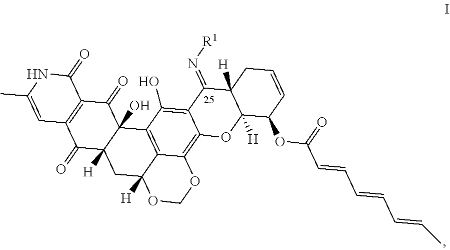

### CROSS-REFERENCE TO RELATED PATENT APPLICATIONS

This application claims the benefit of and priority to U.S. Provisional Patent Application No. 63/249,490, filed on Sep. 28, 2021, the entire contents of which is incorporated herein by reference in its entirety.

### GOVERNMENT RIGHTS

This invention was made with government support under AI109673 and AI142720 awarded by the National Institutes of Health. The government has certain rights in the invention.

### FIELD OF THE TECHNOLOGY

The present technology relates to turbinmicin analogs, compositions and methods of use thereof. The turbinmicin analogs and compositions containing it are useful as antifungals, and show activity even against multi-drug resistant fungal infections.

### BACKGROUND

More than 3-million patients worldwide are afflicted by fungal infections, and this number only continues to rise with increasing at-risk immunocompromised population. Today, only three antifungal drug classes are available for clinical use. The development of new antifungals has been hampered, in part, by the close evolutionary relationship between fungi and their human hosts. In addition to this paucity of drug options, many of the agents exhibit limited efficacy or toxic side-effects. Therefore, despite therapy, patient survival remains unacceptably low. In fact, fungi lead as the cause of infection-related mortality in many cancer and transplant populations. For example, the 90-day survival following a diagnosis of invasive Candida infection approaches 50%. The outcomes are even worse for patients with Aspergillus and other mold infections, with mortality reaching 80-90%. The prevalence of poor outcomes increase further with the recent emergence of pathogens that exhibit resistance to first-line antifungal options. For example, a recent global study of patients with Aspergillus pneumonia identified triazole-resistant Aspergillus sp. in nearly 50% of patients in high-risk groups. Similarly, the utility of echinocandins, first-line agents for treatment of invasive Candida glabrata infection, is currently limited by drug resistance in up to 15% of cases in some medical centers. Most recently, the multidrug resistant “killer fungus”, Candida auris, has emerged and is spreading throughout healthcare facilities. In the United States, C. auris has prompted an urgent threat alert from the Centers for Disease Control and Prevention (CDC).

### SUMMARY

As disclosed herein, the present technology provides novel turbinmicin compounds modified at position 25 (C25) of turbinmicin. In any embodiment of the present technology, the compound may be a compound of Formula I, stereoisomers thereof, tautomers thereof, and/or pharmaceutically acceptable salts thereof:

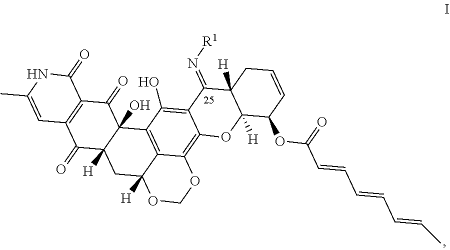

wherein
R¹ may be selected from a substituted or unsubstituted alkyl, cycloalkyl, cycloalkylalkyl, alkenyl, cycloalkenyl, alkynyl, aryl, aralkyl, heterocyclyl, heterocyclylalkyl, heteroaryl, or heteroarylalkyl groups, or OR²; and
R² may be selected from a substituted or unsubstituted alkyl, alkenyl, alkynyl, aralkyl, or heterocyclylalkyl group.

In various aspects and embodiments, the present compound of Formula I provides one or more: (1) a novel scaffold completely different from currently employed antifungal drugs, suggesting a different mechanism of action and thus activity against multidrug resistant fungal infections; (2) amenability to efficient production by the dehydration-condensation of an amine (e.g., R¹NH₂) and the ketone at C25 of turbinmicin with unexpectedly high site-selectivity; (3) high and broad activity against fungal infections; and (4) improved physical and pharmacokinetic properties (e.g., water solubility) compared to turbinmicin.

Pharmaceutical compositions including a compound of Formula I and a pharmaceutically acceptable carrier are also provided. Methods of treating fungal infections by administering an effective amount of a compound of Formula I to a mammal in need thereof are disclosed.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows the structures of various turbinmicin analogs of the present technology prepared by the general procedure of imine formation.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term. In some embodiments, “about” may also refer to plus or minus 5%, 2% or 1% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; sulfates; phosphates; amines; N-oxides; hydrazines; hydrazides; hydrazones; azides (—N₃); amides; ureas; amidines; guanidines; enamines; imides; imines; nitro groups (—NO₂); nitriles (—CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, amidinealkyl, guanidinealkyl, alkoxyalkyl, carboxyalkyl, and the like.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above for alkyl.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic carbon-containing ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, oxadiazolonyl (including 1,2,4-oxazol-5(4H)-one-3-yl), isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic carbon-containing ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent alkenyl groups are alkenylene groups, and so forth. Substituted groups having a single point of attachment to a compound or polymer of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, cycloalkyl, aryl, aralkyl, heterocyclylalkyl or heterocyclyl group as defined herein. Amido groups therefore include but are not limited to carbamoyl groups (—C(O)NH₂) (also referred to as “carboxamido groups”) and formamido groups (—NHC(O)H). In some embodiments, the amide is —NR⁷¹C(O)—(C₁₋₅ alkyl) and the group is termed “alkanoylamino.”

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, cycloalkyl, aryl, aralkyl, heterocyclylalkyl or heterocyclyl group as defined herein. In some embodiments, the amine is NH₂, alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. It will be understood that amines may exist in protonated forms in certain aqueous solutions or mixtures and are examples of charged functional groups herein.

The term “carboxyl” or “carboxylate” as used herein refers to a —COOH group or its ionized salt form. As such, it will be understood that carboxyl groups are examples of charged functional groups herein.

The term “ester” as used herein refers to —COOR⁷⁰ and —C(O)O-G groups. R⁷⁰ is a substituted or unsubstituted alkyl, cycloalkyl, alkenyl, aryl, aralkyl, heterocyclylalkyl or heterocyclyl group as defined herein. G is a carboxylate protecting group. As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3^{rd} Edition, 1999). Which can be added or removed using the procedures set forth therein and which is hereby incorporated by reference in its entirety and for any and all purposes as if fully set forth herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, aryl aralkyl, heterocyclyl or heterocyclylalkyl group as defined herein. It will be understood that guanidines may exist in protonated forms in certain aqueous solutions or mixtures and are examples of charged functional groups herein.

The term “hydroxyl” as used herein can refer to —OH or its ionized form, —O⁻. A “hydroxyalkyl” group is a hydroxyl-substituted alkyl group, such as HO—CH₂—.

The term “imidazolyl” as used herein refers to an imidazole group or the salt thereof. An imidazolyl may be protonated in certain aqueous solutions or mixtures, and is then termed an “imidazolate.”

The term “phosphate” as used herein refers to —OPO₃H₂ or any of its ionized salt forms, —OPO₃HR⁸⁴ or —OPO₃R⁸⁴R⁸⁵ wherein R⁸⁴ and R⁸⁵ are independently a positive counterion, e.g., Na⁺, K⁺, ammonium, etc. As such, it will be understood that phosphates are examples of charged functional groups herein.

The term “pyridinyl” refers to a pyridine group or a salt thereof. A pyridinyl may be protonated in certain aqueous solutions or mixtures, and is then termed a “pyridinium group”.

The term “sulfate” as used herein refers to —OSO₃H or its ionized salt form, —OSO₃R⁸⁶ wherein R⁸⁶ is a positive counterion, e.g., Na⁺, K⁺, ammonium, etc. As such, it will be understood that sulfates are examples of charged functional groups herein.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, and R⁸² are each independently a substituted or unsubstituted alkyl, cycloalkyl, alkenyl, aryl aralkyl, heterocyclyl or heterocyclylalkyl group as defined herein. In some embodiments the sulfide is an alkylthio group, —S-alkyl. R⁸³ includes H or, when the sulfonyl is ionized (i.e., as a sulfonate), a positive counterion, e.g., Na⁺, K⁺, ammonium or the like. As such, it will be understood that sulfonyls are examples of charged functional groups herein.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

“Treating” within the context of the instant technology, means alleviation, in whole or in part, of symptoms associated with a disorder or disease, or slowing, inhibition or halting of further progression or worsening of those symptoms, or prevention or prophylaxis of the disease or disorder in a subject at risk for developing the disease or disorder. For example, within the context of treating fungal infections, successful treatment may include reduction or eradication of the pathogenic fungus, from the body; clinical benefit; an alleviation of symptoms, such as a reduction or elimination of rash, itching, chafing, burning, throat thrush, redness, soreness, fever, cough, night sweats, weight loss, wheezing, and shortness of breath.

As used herein, an “effective amount” of a compound of the present technology refers to an amount of the compound that alleviates, in whole or in part, symptoms associated with a disorder or disease, or slows or halts of further progression or worsening of those symptoms, or prevents or provides prophylaxis for the disease or disorder in a subject at risk for developing the disease or disorder. Those skilled in the art are readily able to determine an effective amount. For example, one way of assessing an effective amount for a particular disease state is by simply administering a compound of the present technology to a patient in increasing amounts until progression of the disease state is decreased or stopped or reversed. An “effective amount” of a compound of the present technology also refers to an amount of the compound that, for example, reduces a population of fungi where the fungal population may be outside a subject (e.g., in a media in a container).

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereoisomeric or geometric isomeric forms, it should be understood that the technology encompasses any tautomeric, conformational isomeric, stereoisomeric and/or geometric isomeric forms of the compounds having one or more of the utilities described herein, as well as mixtures of these various different forms.

Stereoisomers of compounds (also known as optical isomers) include all chiral, diastereomeric, and racemic forms of a structure, unless the specific stereochemistry is expressly indicated. Thus, compounds disclosed herein include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, imines may exhibit the following isomeric forms, which are referred to as tautomers of each other:

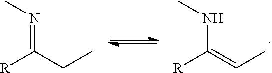

Because of the limits of representing compounds by structural formulas, it is to be understood that all chemical formulas of the compounds described herein represent all tautomeric forms of compounds and are within the scope of the present technology.

In one aspect, the present technology provides turbinmicin analogs modified at carbon-25 (C25). Thus, the present technology provides compounds of Formula I, stereoisomers thereof, tautomers thereof and/or pharmaceutically acceptable salts thereof:

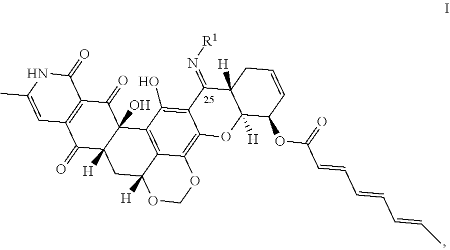

wherein
R¹ may be selected from a substituted or unsubstituted alkyl, cycloalkyl, cycloalkylalkyl, alkenyl, cycloalkenyl, alkynyl, aryl, aralkyl, heterocyclyl, heterocyclylalkyl, heteroaryl, or heteroarylalkyl groups, or OR²; and
R² may be selected from a substituted or unsubstituted alkyl, alkenyl, alkynyl, aralkyl, or heterocyclylalkyl group.

In any embodiments of compounds of Formula I disclosed herein, R¹ may be a substituted or unsubstituted alkyl, alkenyl, alkynyl, aralkyl, heteroarylalkyl, heterocyclylalkyl, or OR² wherein R² is a substituted or unsubstituted alkyl. In any embodiments, R¹ may be a substituted or unsubstituted alkyl group. In any embodiments, R¹ may be a substituted or unsubstituted alkenyl group. In any embodiments, R¹ may be a substituted or unsubstituted alkynyl group. In any embodiments, R¹ may be a substituted or unsubstituted aryl or aralkyl group. In any embodiments, R¹ may be a substituted or unsubstituted aralkyl group. In any embodiments, R¹ may be a substituted or unsubstituted heterocyclyl or heterocyclylalkyl group. In any embodiments, R¹ may be a substituted or unsubstituted heterocyclylalkyl group. In any embodiments, R¹ may be a substituted or unsubstituted heteroarylalkyl group. In any embodiments, R¹ may be OR² and R² may be a substituted or unsubstituted alkyl group.

In any embodiments, R¹ and/or R² may be independently unsubstituted or substituted with one or more substituents selected from halo, OH, CN, COOH, COOR³, C(O)R³, NO₂, NR⁴R⁵, or C(O)NR⁴R⁵ wherein R³ at each occurrence is independently H or an unsubstituted alkyl, alkenyl, or aralkyl group; and R⁴ and R⁵ at each occurrence are independently H or an unsubstituted alkyl, alkenyl, or aralkyl group. In any embodiments, R⁴ and R⁵ at each occurrence are independently H or an unsubstituted alkyl group, e.g., a C₁₋₆ alkyl group such as a methyl group.

In any embodiments, R¹ may be selected from a C₁₋₆ alkyl, C₂₋₆ alkenyl, or C₂₋₆ alkynyl group, optionally substituted with a substituent selected from OH or NR⁴R⁵ wherein R⁴ and R⁵ may independently have any values described herein.

In any embodiments, R¹ may be selected from aralkyl, heterocyclylalkyl, or heteroarylalkyl groups, each of which is optionally substituted with one or two C₁₋₆ alkyl, aryl, or heteroaryl groups. In any embodiments, R¹ may be a substituted or unsubstituted benzyl, phenethyl or phenpropyl group. In any embodiments, R¹ may be a heterocyclylalkyl group selected from morpholinyl-C₁₋₆ alkyl, piperidinyl-C₁₋₆ alkyl, piperazinyl-C₁₋₆ alkyl, pyrrolidinyl-C₁₋₆ alkyl, thiazolidinyl-C₁₋₆ alkyl, or thiazolidinyl-1,1-dioxide-C₁₋₆ alkyl, thiomorpholinyl-C₁₋₆ alkyl, or thiomorpholinyl-1,1-dioxide-C₁₋₆ alkyl; or R¹ may be a heteroarylalkyl selected from pyridinyl-C₁₋₆ alkyl. In any embodiments, R¹ may be a heteroarylalkyl group, e.g., pyridinyl-C₁₋₆ alkyl. In any embodiments, R¹ may be a substituted or unsubstituted piperazinyl-C₁₋₆ alkyl. In any embodiments, R¹ may be piperazinyl-C₁₋₆ alkyl substituted with one or two C₁₋₆ alkyl, e.g., one or two methyl groups on the piperazinyl ring. In any embodiments, R¹ may be piperazinyl-C₁₋₆ alkyl substituted with a single C₁₋₆ alkyl group on a piperazinyl ring nitrogen (to provide a tertiary amine), e.g., a methyl or ethyl group. In any embodiments, R¹ may be piperazinyl-C₁₋₆ alkyl substituted with one or two aryl or heteroaryl groups. In any embodiments, R¹ may be morpholinyl-C₁₋₆ alkyl substituted with one or two C₁₋₆ alkyl, e.g., one or two methyl groups on the morpholinyl ring.

Surprisingly, compounds of Formula I may be prepared regiospecifically from the densely functionalized natural product, turbinmicin (see WO2020/146155, incorporated by reference herein in its entirety) in good yield and without epimerization any stereocenter. Thus, a compound of Formula II (turbinmicin) may be reacted with R¹NH₂ in the presence of an acid catalyst and a suitable solvent to provide a compound of Formula I. R¹ may have any of the values disclosed herein.

In another aspect the present technology provides a pharmaceutical composition including compound of Formula I as described herein and a pharmaceutically acceptable carrier. The pharmaceutical compositions of any embodiment herein may be formulated for oral, parenteral, nasal, topical administration or any of the routes discussed herein. In any embodiment herein, the pharmaceutical composition may include an effective amount of a compound of any embodiment of the present technology. The effective amount may be an effective amount for treating a fungal infection, including those caused by any of the fungi disclosed herein. In any embodiments, the effective amount of compound may be an effective amount for treating any infection due to drug-resistant fungi, including those disclosed herein (see below).

The present technology provides methods of treating a fungal infection comprising administering an effective amount of compound of Formula I, or a pharmaceutical composition as described herein to a mammal in need thereof. The mammal may be, e.g., a human, primate (e.g. monkey, chimpanzee, ape), cat, dog, pig, mouse, rat, horse, sheep, among others. In any embodiment described herein, the mammal may be human. The infection may occur, e.g., in the skin, mouth, pharynx, esophagus, toenails, fingernails, urogenital tract, or lungs, or may be systemic, in, e.g., immunocompromised patients. In any embodiment of the present methods, the fungal infection may be caused by one or both of Candida, Fusarium, Scedosporium, Rhizopus, Mucor, Apophysomyces, Lichteimia, Cynninghamella, or Aspergillus. In any embodiments of the present methods, the fungal infection may be caused by Aspergillus, such as Aspergillus fumigatus, or it may be caused by Candida, e.g., Candida albicans, Candida auris, Candida enolase, Candida tropicalis, Candida glabrata, Candida krusei, Candida parapsilosis, Candida stellatoidea, Candida parakawsei, Candida lusitaniae, Candida pseudotropicalis, and Candida guilliermondi. In any embodiments of the present methods, the fungal infection may be caused by Candida albicans, Candida glabrata, Candida auris, Candida tropicalis, Rhizopus delemar, Mucor circinelloides, Apophysomyces elegans, Lichteimia corymbiferea, Aspergillus fumigatus, and drug-resistant strains thereof. In any embodiment described herein, the fungal infection may be caused by one or more of drug-resistant fungi, such as, but not limited to, Aspergillus fumigatus (e.g., 11628), Candida albicans, Candida glabrata (e.g., 4720), Candida auris (e.g., B 11211). Similarly, the compounds and compositions described herein may be used for therapy, such as for treatment of fungal infections such as any of those described herein, or for use in the manufacture of a medicament for any such treatments.

In another aspect, the present technology provides pharmaceutical compositions of compound of Formula I with a second antifungal agent (or combination of agents) different from compound of Formula I, including but not limited to azoles, echinocandins, or polyenes, as well as methods of using the same. Antifungal agents include drugs which demonstrate clinical benefit in treatment of fungal infections in a mammal, including a human. Suitable second antifungal agents include but are not limited to one or more of amphotericin B, flucytosine, fluconazole, isavuconazole, micafungin, voriconazole, posaconazole and forazoline. In any embodiment described herein, an effective amount of a compound as described herein (e.g., compound of Formula I), a salt thereof or a pharmaceutical composition comprising the compound and a pharmaceutically acceptable carrier, may be administered to a mammal in need thereof, wherein the second antifungal agent(s) is/are administered to the mammal in need thereof simultaneously, sequentially or separately with a compound as described herein, or any embodiment of the pharmaceutical composition as describe herein.

The instant technology also provides for compositions and medicaments including a compound disclosed herein and a pharmaceutically acceptable carrier. Such compositions may be prepared by mixing one or more compounds of the present technology, pharmaceutically acceptable salts thereof or stereoisomers thereof, with pharmaceutically acceptable carriers, excipients, binders, diluents or the like to treat fungal infections. The compounds and compositions of the present technology may be used to prepare formulations and medicaments that treat a variety of fungal infections, e.g., Candida and Aspergillus as disclosed herein. Such compositions can be in the form of, for example, granules, powders, tablets, capsules, creams, ointments, syrup, suppositories, injections, emulsions, elixirs, suspensions or solutions. The instant compositions can be formulated for various routes of administration, for example, by oral, parenteral, topical, injection, rectal, nasal, vaginal, or via implanted reservoir. Parenteral or systemic administration includes, but is not limited to, subcutaneous, intravenous, intraperitoneally, intramuscular, intrathecal, intracranial, and intracerebroventricular injections. The following dosage forms are given by way of example and should not be construed as limiting the instant technology.

For oral, buccal, and sublingual administration, powders, suspensions, granules, tablets, pills, capsules, gelcaps, and caplets are acceptable as solid dosage forms. These can be prepared, for example, by mixing one or more compounds disclosed herein, or pharmaceutically acceptable salts or stereoisomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions, which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology also may be formulated as a composition for topical administration (e.g., vaginal cream). These formulations may contain various excipients known to those skilled in the art. Suitable excipients may include, but are not limited to, cetyl esters wax, cetyl alcohol, white wax, glyceryl monostearate, propylene glycol monostearate, methyl stearate, benzyl alcohol, sodium lauryl sulfate, glycerin, mineral oil, water, carbomer, ethyl alcohol, acrylate adhesives, polyisobutylene adhesives, and silicone adhesives.

The composition may be in the form of a vaginal cream containing the composition of matter as set forth herein present in a nonliquefying base. The nonliquefying base may contain various inactive ingredients such as, for example, cetyl esters wax, cetyl alcohol, white wax, glyceryl monostearate, propylene glycol monostearate, methyl stearate, benzyl alcohol, sodium lauryl sulfate, glycerin, and mineral oil. Such composition may be formulated similar to PREMARIN® Vaginal Cream made commercially available by Wyeth-Ayerst Laboratories.

Dosage units for rectal administration may be prepared in the form of suppositories which may contain the composition of matter in a mixture with a neutral fat base, or they may be prepared in the form of gelatin-rectal capsules which contain the active substance in a mixture with a vegetable oil or paraffin oil.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of inventive compounds by inhalation.

Ordinarily, an aqueous aerosol is made by formulating an aqueous solution or suspension of the compound together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins such as serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver compounds of the present technology.

Aerosols containing compounds for use according to the present technology are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch. Delivery of aerosols of the present technology using sonic nebulizers is advantageous because nebulizers minimize exposure of the agent to shear, which can result in degradation of the compound.

For nasal administration, the pharmaceutical formulations and medicaments may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the compounds may be formulated in oily solutions or as a gel. For administration of nasal aerosol, any suitable propellant may be used including compressed air, nitrogen, carbon dioxide, or a hydrocarbon based low boiling solvent.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant technology.

A therapeutically effective amount of a compound of the present technology may vary depending upon the route of administration and dosage form. Effective amounts of such compounds typically fall in the range of about 0.01 up to about 100 mg/kg/day, or about 0.05 to about 50 mg/kg/day, and more typically in the range of about 0.1 up to 5 mg/kg/day or 10 mg/kg/day. Typically, the compound or compounds of the instant technology are selected to provide a formulation that exhibits a high therapeutic index. The therapeutic index is the dose ratio between toxic and therapeutic effects and can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology is further illustrated by the following examples, which should not be construed as limiting in any way.

### EXAMPLES

### Materials and General Experimental Procedures

Proton nuclear magnetic resonance (H NMR) spectra were recorded on Bruker AV400, AV500, and AV600 spectrometers at 25° C. Proton chemical shifts are expressed as parts per million (ppm, 6 scale) and are referenced to the residual solvent peak (CDCl₃, δ 7.26; CD₃OD δ 3.29). HRMS data were acquired with a Bruker MaXis 4G QTOF mass spectrometer.

### Example 1: Chemical Syntheses of the Turbinmicin Analogs of the Present Technology

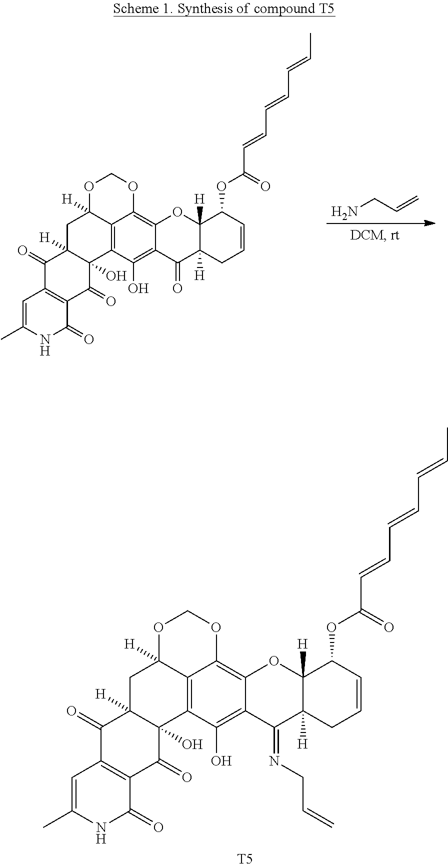

T5 was synthesize according to Scheme 1. Briefly, to a solution of Turbinmicin (10 mg, 0.016 mmol) in dry DCM (1 mL) at 25° C. was added allylamine (50 eq.). After stirring at room temperature for 2 h, the reaction mixture was concentrated. The resulting mixture was purified by reversed phase HPLC to give T5 in 90% yield. ¹H NMR (600 MHz, CDCl₃) δ 7.38 (dd, J=14.9, 11.3 Hz, 1H), 6.54 (dd, J=14.9, 11.7 Hz, 1H), 6.50 (s, 1H), 6.22 ((dd, J=14.9, 11.3 Hz, 1H), 6.17 (dd, J=14.9, 11.7 Hz, 1H), 6.01 (m, 1H), 5.97 (m, 1H), 5.94 (d, J=15.0, 1H), 5.86 (m, 1H), 5.70 (m, 1H), 5.62 (m, 1H), 5.34 (m, 1H), 5.33 (d, J=5.7 Hz, 1H), 5.28 (d, J=5.7 Hz, 1H), 5.18 (m, 1H), 5.12 (m, 1H), 4.53 (m, 1H), 4.21 (d, J=16.5 Hz, 1H), 4.13 (d, J=16.5 Hz, 1H), 3.31 (m, 1H), 3.26 (m, 1H), 2.66 (m, 2H), 2.36 (s, 3H), 2.23 (m, 1H), 2.14 (m, 1H), 1.83 (dd, J=6.8, 1.6 Hz, 3H); HRMS (ESI⁺) calc. for C₃₇H₃₅N₂O₁₀⁺ ([M+H]⁺) 667.2286, found 667.2287.

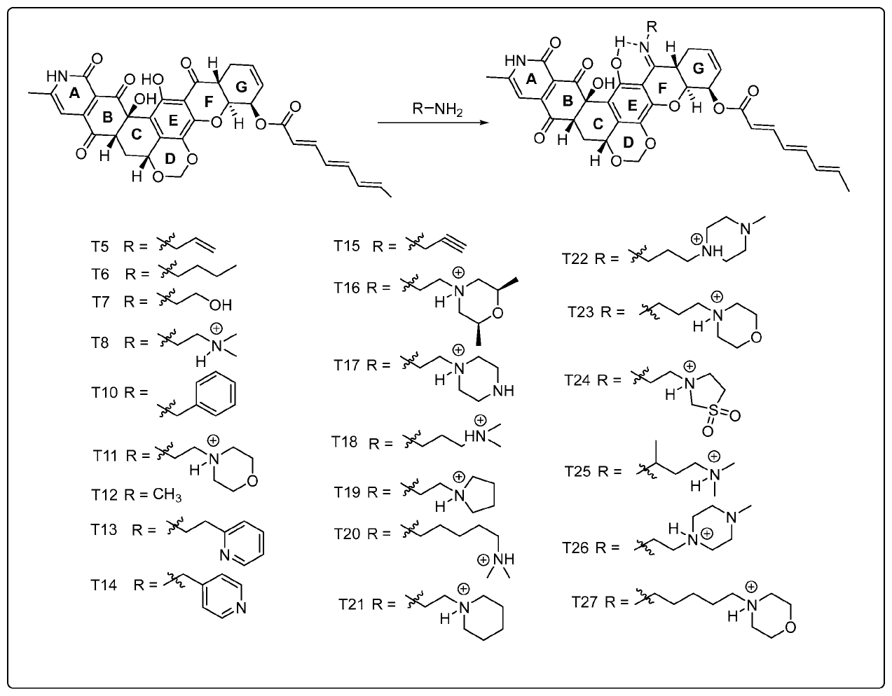

Compounds of Examples 2-22 were synthesized following the procedure of Example 1. See also FIG. 1.

### Example 2: Synthesis of Compound T6

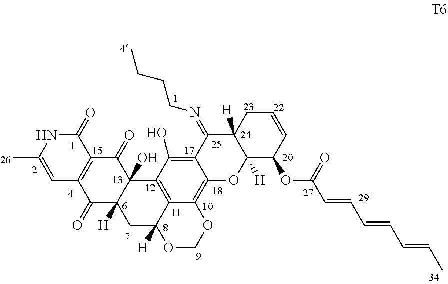 [SMILES: C/C=C/C=C/C=C/C(=O)O[C@@H]1C=CC[C@H]2/C(=N/CCCC)c3c(O)c4c5c(c3O[C@H]12)OCO[C@@H]5C[C@@H]1C(=O)c2cc(C)[nH]c(=O)c2C(=O)[C@]41O]

T6. ¹H NMR (600 MHz, CDCl₃) δ 7.40 (dd, J=14.9, 11.3 Hz, 1H), 6.55 (dd, J=14.9, 11.7 Hz, 1H), 6.53 (s, 1H), 6.23 ((dd, J=14.9, 11.3 Hz, 1H), 6.17 (dd, J=14.9, 11.7 Hz, 1H), 6.01 (m, 1H), 5.97 (m, 1H), 5.94 (d, J=15.0, 1H), 5.72 (m, 1H), 5.63 (m, 1H), 5.35 (d, J=5.7 Hz, 1H), 5.29 (d, J=5.7 Hz, 1H), 5.11 (m, 1H), 4.56 (m, 1H), 3.51 (m, 1H), 3.42 (m, 1H), 3.31 (t, J=5.8 Hz, 1H), 3.24 (m, 1H), 2.60 (m, 2H), 2.36 (s, 3H), 2.26-2.15 (m, 2H), 1.84 (dd, J=6.8, 3H), 1.68-1.57 (m, 2H), 1.36 (m, 2H), 0.89 (t, J=6.0, 3H); HRMS (ESI⁺) calc. for C₃₈H₃₉N₂O₁₀⁺ ([M+H]⁺) 683.2599, found 683.2587.

### Example 3: Synthesis of Compound T7

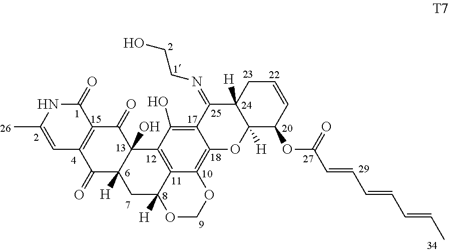 [SMILES: C/C=C/C=C/C=C/C(=O)O[C@@H]1C=CC[C@H]2/C(=N/CCO)c3c(O)c4c5c(c3O[C@H]12)OCO[C@@H]5C[C@@H]1C(=O)c2cc(C)[nH]c(=O)c2C(=O)[C@]41O]

T7. ¹H NMR (600 MHz, CDCl₃:CD₃OD 1:1) δ 7.35 (dd, J=14.9, 10.7 Hz, 1H), 6.53 (dd, J=14.9, 10.7 Hz, 1H), 6.36 (s, 1H), 6.20 ((dd, J=14.9, 10.7 Hz, 1H), 6.14 (dd, J=14.9, 10.7 Hz, 1H), 5.94 (dd, J=14.8, 7.1 Hz, 1H), 5.90 (m, 1H), 5.89 (d, J=14.9 Hz, 1H), 5.65 (d, J=10.4 Hz, 1H), 5.56 (m, 1H), 5.26 (d, J=5.7 Hz, 1H), 5.24 (d, J=5.7 Hz, 1H), 5.12 (dd, J=9.7, 6.5 Hz, 1H), 4.55 (m, 1H), 3.76 (m, 1H), 3.72 (m, 1H), 3.64 (m, 1H), 3.53 (m, 1H), 3.38 (m, 1H), 3.27 (m, 1H), 2.72 (m, 1H), 2.56 (m, 1H), 2.36 (s, 3H), 2.28 (m, 1H), 2.06 (m, 1H), 1.79 (d, J=6.8 Hz, 3H); HRMS (ESI⁺) calc. for C₃₆H₃₅N₂O₁₁⁺ ([M+H]⁺) 671.2235, found 671.2231.

### Example 4: Synthesis of Compound T8

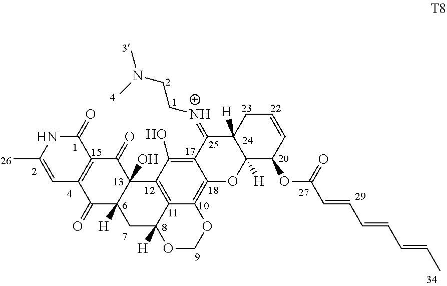 [SMILES: C/C=C/C=C/C=C/C(=O)O[C@@H]1C=CC[C@H]2/C(=[NH+]/CCN(C)C)c3c(O)c4c5c(c3O[C@H]12)OCO[C@@H]5C[C@@H]1C(=O)c2cc(C)[nH]c(=O)c2C(=O)[C@]41O]

T8. ¹H NMR (500 MHz, CD₃OD) 7.37 (dd, J=14.9, 11.2 Hz, 1H), 6.60 (dd, J=14.9, 11.2 Hz, 1H), 6.36 (s, 1H), 6.26 (dd, J=14.9, 11.2 Hz, 1H), 6.18 (dd, J=14.9, 11.2 Hz, 1H), 5.99 (dd, J=14.9, 7.1 Hz, 1H), 5.93 (d, J=14.9 Hz, 1H), 5.87 (m, 1H), 5.61 (d, J=10.4 Hz, 1H), 5.56 (m, 1H), 5.29 (d, J=5.7 Hz, 1H), 5.27 (d, J=5.7 Hz, 1H), 5.21 (dd, J=10.0, 6.4 Hz, 1H), 4.48 (m, 1H), 3.99 (dd, J=16.0, 6.4 Hz, 1H), 3.50 (m, 1H), 3.49 (dd, J=16.0, 5.8 Hz, 1H), 3.45 (m, 1H), 3.45 (m, 1H), 3.06 (t, J=6.1 Hz, 1H), 2.78 (m, 1H), 2.69 (m, 1H), 2.60 (m, 1H), 2.91 (s, 6H), 2.21 (s, 1H), 2.29 (s, 3H), 1.82 (d, J=6.8 Hz, 3H); HRMS (ESI⁺) calc. for C₃₈H₄₀N₃O₁₀⁺ (M⁺) 698.2705, found 698.2708.

### Example 5: Synthesis of Compound T10

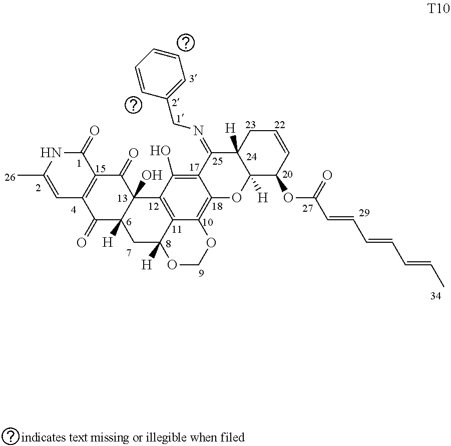 [SMILES: C/C=C/C=C/C=C/C(=O)O[C@@H]1C=CC[C@H]2/C(=N/Cc3ccccc3)c3c(O)c4c5c(c3O[C@H]12)OCO[C@@H]5C[C@@H]1C(=O)c2cc(C)[nH]c(=O)c2C(=O)[C@]41O]

T10. ¹H NMR (500 MHz, CD₃OD) δ 7.37 (dd, J=14.9, 11.2 Hz, 1H), 7.29 (t, J=7.7, 2H), 7.26 (s, 1H), 7.20 (t, J=7.7 Hz, 1H), 7.16 (d, J=7.7 Hz, 2H), 6.53 (dd, J=14.9, 11.2 Hz, 1H), 6.34 (s, 1H), 6.20 (dd, J=14.9, 11.2 Hz, 1H), 6.14 (dd, J=14.9, 11.2 Hz, 1H), 5.94 (dd, J=14.9, 7.1 Hz, 1H), 5.88 (d, J=14.9 Hz, 1H), 5.84 (m, 1H), 5.63 (d, J=10.4 Hz, 1H), 5.55 (m, 1H), 5.31-5.24 (m, 2H), 5.21 (t, J=8.4 Hz, 1H), 4.76 (d, J=15.5 Hz, 1H), 4.64 (d, J=15.5 Hz, 1H), 4.47 (m, 1H), 3.40 (t, J=9.0 Hz, 1H), 3.26 (m, 1H), 2.72 (m, 2H), 2.60 (m, 1H), 2.25 (s, 3H), 2.05 (m, 1H), 1.80 (dd, J=6.9, 1.5 Hz, 3H); HRMS (ESI⁺) calc. for C₄₁H₃₇N₂O₁₀⁺ ([M+H]⁺) 717.2443, found 717.2445.

### Example 6: Synthesis of Compound T11

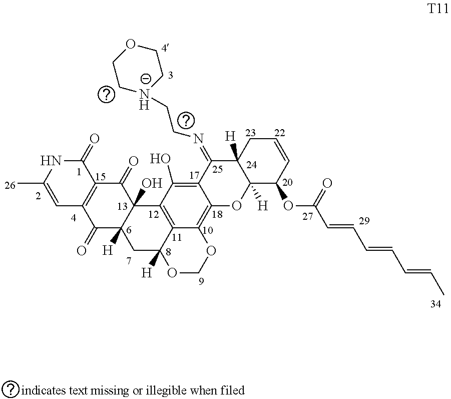

T11. ¹H NMR (500 MHz, CD₃OD) δ 7.36 (dd, J=14.9, 11.3 Hz, 1H), 6.55 (dd, J=14.9, 10.6 Hz, 1H), 6.35 (s, 1H), 6.18 (dd, J=14.9, 11.3 Hz, 1H), 6.10 (dd, J=14.9, 11.2 Hz, 1H), 5.96 (dd, J=14.9, 7.1 Hz, 1H), 5.90 (d, J=14.9 Hz, 1H), 5.87 (m, 1H), 5.60 (d, J=10.0 Hz, 1H), 5.55 (s, 1H), 5.27 (d, J=5.7 Hz, 1H), 5.25 (d, J=5.7 Hz, 1H), 5.17 (dd, J=9.8, 6.4 Hz, 1H), 4.49 (m, 1H), 3.78 (dt, J=14.8, 6.4 Hz, 1H), 3.70 (m, 4H), 3.61 (dt, J=14.8, 5.8 Hz, 1H), 3.46 (m, 1H), 3.27 (m, 1H), 2.90-2.82 (m, 2H), 2.67 (m, 4H), 2.73 (m, 1H), 2.57 (m, 1H), 2.27 (s, 3H), 2.25 (m, 1H), 1.89 (m, 1H), 1.79 (dd, J=6.9, 1.5 Hz, 3H); HRMS (ESI⁺) calc. for C₄₀H₄₂N₃₀O₁₁⁺ (M⁺) 740.2814, found 740.2806.

### Example 7: Synthesis of Compound T12

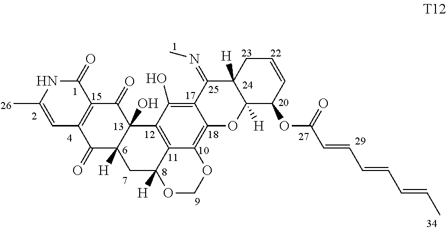 [SMILES: C/C=C/C=C/C=C/C(=O)O[C@@H]1C=CC[C@H]2/C(=N/C)c3c(O)c4c5c(c3O[C@H]12)OCO[C@@H]5C[C@@H]1C(=O)c2cc(C)[nH]c(=O)c2C(=O)[C@]41O]

T12. ¹H NMR (400 MHz, CDCl₃) δ 7.38 (dd, J=15.2, 11.4 Hz, 1H), 6.55 (dd, J=14.9, 11.7 Hz, 1H), 6.51 (s, 1H), 6.22 (dd, J=14.9, 11.3 Hz, 1H), 6.16 (dd, J=14.9, 11.7 Hz, 1H), 5.99 (m, 1H), 5.94 (m, 1H), 5.93 (d, J=15.0, 1H), 5.72 (m, 1H), 5.63 (s, 1H), 5.32 (d, J=5.7 Hz, 1H), 5.27 (d, J=5.7 Hz, 1H), 5.12 (m, 1H), 4.54 (m, 1H), 3.33-3.15 (m, 5H), 2.64 (m, 2H), 2.46 (s, 3H), 2.28 (s, 1H), 2.23 (s, 1H), 2.14 (m, 1H), 1.83 (d, J=6.8 Hz, 3H); HRMS (ESI⁺) calc. for C₃₅H₃₃N₂O₁₀⁺ ([M+H]⁺) 641.2122, found 641.2130.

### Example 8: Synthesis of Compound T13

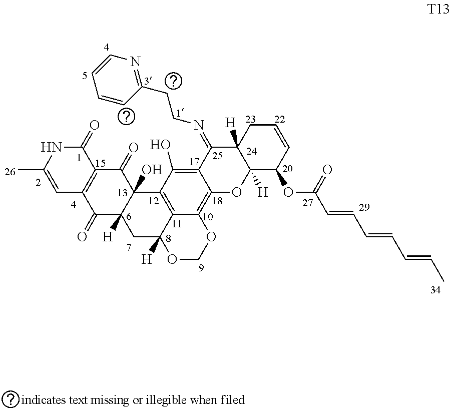 [SMILES: C/C=C/C=C/C=C/C(=O)O[C@@H]1C=CC[C@H]2/C(=N/CCc3ccccn3)c3c(O)c4c5c(c3O[C@H]12)OCO[C@@H]5C[C@@H]1C(=O)c2cc(C)[nH]c(=O)c2C(=O)[C@]41O]

T13. ¹H NMR (400 MHz, CDCl₃) δ 8.53 (m, 1H), 7.62 (t, J=7.6 Hz, 1H), 7.38 (dd, J=15.2, 11.3 Hz, 1H; H-29), 7.19 (t, J=7.6 Hz, 1H), 7.07 (d, J=7.6 Hz, 1H), 6.72 (s, 1H), 6.55 (dd, J=14.8, 10.7 Hz, 1H; H-31), 6.22 (dd, J=14.9, 11.3 Hz, 1H; H-30), 6.16 (dd, J=14.9, 11.7 Hz, 1H; H-32), 6.02-5.84 (m, 4H), 5.66 (m, 1H; H-21), 5.50 (s, 1H; H-20), 5.32 (d, J=5.7 Hz, 1H; H-9a), 5.27 (d, J=5.7 Hz, 1H; H-9b), 5.11 (m, 1H; H-8), 4.21 (m, 1H; H-19), 3.99 (m, 1H; H-1′a), 3.85 (m, 1H; H-1′b), 3.72 (m, 1H), 3.32 (m, 1H; H-6), 3.22-3.07 (m, 2H), 2.60 (m, 2H; H₂-7), 2.48 (s, 3H; H₃-26), 2.33 (m, 1H), 2.04 (m, 1H), 1.84 (d, J=6.8 Hz, 3H, H₃-34); HRMS (ESI⁺) calc. for C₄₁H₃₈N₃O₁₀⁺ ([M+H]⁺) 732.2552, found 732.2547.

### Example 9: Synthesis of Compound T14

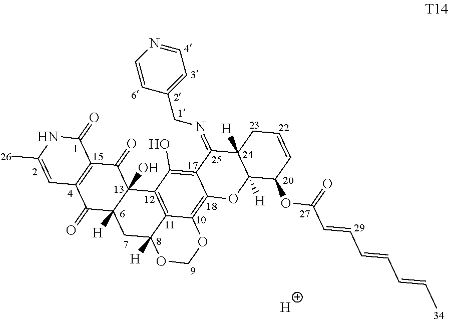

T14. ¹H NMR (400 MHz, Chloroform-d) δ 9.07 (d, J=5.5 Hz, 2H), 7.84 (d, J=5.5 Hz, 2H), 7.37 (dd, J=15.2, 11.3 Hz, 1H), 6.56 (dd, J=14.9, 11.7 Hz, 1H), 6.51 (s, 1H), 6.22 (dd, J=14.9, 11.3 Hz, 1H), 6.17 (dd, J=14.9, 11.7 Hz, 1H), 5.97 (m, 1H), 5.92 (m, 1H), 5.91 (d, J=15.0, 1H), 5.69 (d, J=10.5 Hz, 1H), 5.59 (m, 1H), 5.37 (d, J=5.7 Hz, 1H), 5.32 (d, J=5.7 Hz, 1H), 5.24 (m, 1H), 5.00 (d, 1H), 4.90 (d, 1H), 4.55 (m, 1H), 3.43 (d, J=7.7 Hz, 1H), 3.32 (s, 1H), 2.96 (m, 1H), 2.78 (m, 1H), 2.44 (s, 3H), 2.28 (m, 1H), 2.07 (m, 1H), 1.85 (d, J=6.8, 3H); HRMS (ESI⁺) calc. for C₄₀H₃₆N₃O₁₀⁺ ([M+H]⁺) 718.2395, found 718.2384.

### Example 10: Synthesis of Compound T15

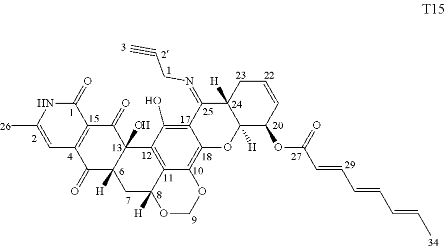 [SMILES: C#CC/N=C1\c2c(O)c3c4c(c2O[C@@H]2[C@H](OC(=O)/C=C/C=C/C=C/C)C=CC[C@@H]12)OCO[C@@H]4C[C@@H]1C(=O)c2cc(C)[nH]c(=O)c2C(=O)[C@]31O]

T15. ¹H NMR (500 MHz, CDCl₃:CD₃OD 1.1) δ 7.39 (dd, J=15.2, 11.3 Hz, 1H), 6.55 (dd, J=14.9, 10.7 Hz, 1H), 6.54 (s, 1H), 6.23 (dd, J=14.9, 11.2 Hz, 1H), 6.18 (dd, J=14.9, 11.2 Hz, 1H), 5.98 (dd, J=14.9, 7.1 Hz, 1H), 5.94 (m, 1H), 5.93 (d, J=14.9 Hz, 1H), 5.61 (d, J=10.6 Hz, 1H), 5.62 (m, 1H), 5.36 (d, J=5.8 Hz, 1H), 5.30 (d, J=5.8 Hz, 1H), 5.18 (m, 1H), 4.54 (m, 1H), 4.26 (s, 2H), 3.37-3.29 (m, 2H), 2.66 (m, 1H), 2.47 (s, 3H), 2.35 (m, 1H), 2.31 (m, 1H), 2.24 (s, 1H), 2.14 (m, 1H), 1.84 (d, J=6.7 Hz, 3H); HRMS (ESI⁺) calc. for C₃₇H₃₃N₂O₁₀⁺ ([M+H]⁺) 655.2130, found 655.2126.

### Example 11: Synthesis of Compound T16

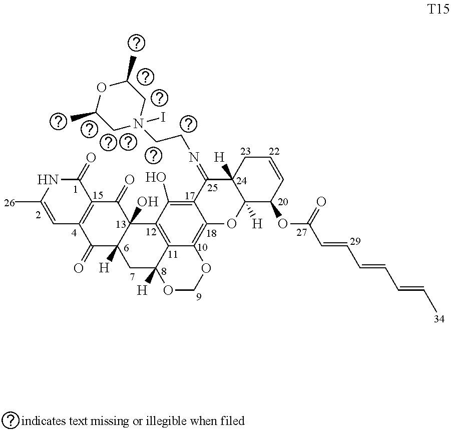

T16. ¹H NMR (500 MHz, CDCl₃:CD₃OD 1:1) δ 7.38 (dd, J=15.2, 11.3 Hz, 1H), 6.54 (dd, J=14.9, 10.7 Hz, 1H), 6.50 (s, 1H), 6.21 (dd, J=14.9, 11.2 Hz, 1H), 6.16 (dd, J=14.9, 11.2 Hz, 1H), 5.98 (dd, J=14.9, 7.1 Hz, 1H), 5.94 (m, 1H), 5.93 (d, J=14.9 Hz, 1H), 5.71 (d, J=10.4 Hz, 1H), 5.61 (m, 1H), 5.34 (d, J=5.7 Hz, 1H), 5.28 (d, J=5.8 Hz, 1H), 5.10 (m, 1H), 4.56 (m, 1H), 3.66 (m, 1H), 3.61 (d, J=5.8 Hz, 2H), 3.58 (m, 1H), 3.28 (m, 2H), 2.80-2.53 (m, 7H), 2.45 (s, 3H), 2.25 (m, 1H); 2.16 (m, 1H), 1.86 (m, 1H), 1.83 (d, J=7.6, 3H), 1.11 (d, J=5.8 Hz, 3H), 1.10 (d, J=5.8 Hz, 3H); HRMS (ESI⁺) calc. for C₄₂H₄₆N₃O₁₁⁺ (M⁺) 768.3127, found 768.3132.

### Example 12: Synthesis of Compound T17

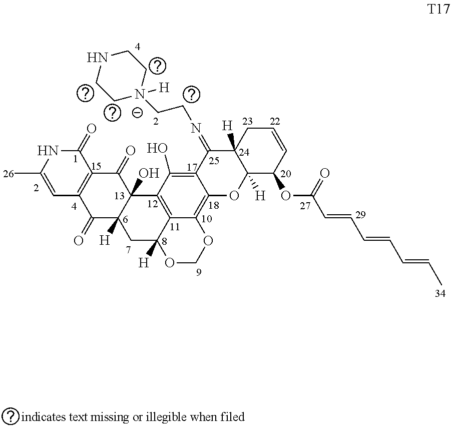

T17. ¹H NMR (400 MHz, CDCl₃:CD₃OD 1:1) δ 7.36 (dd, J=15.2, 11.3 Hz, 1H), 6.54 (dd, J=14.9, 10.7 Hz, 1H), 6.35 (s, 1H), 6.22 (dd, J=14.9, 11.2 Hz, 1H), 6.14 (dd, J=14.9, 11.2 Hz, 1H), 5.97 (dd, J=14.9, 7.1 Hz, 1H), 5.92 (m, 1H), 5.88 (d, J=14.9 Hz, 1H), 5.66 (d, J=10.4 Hz, 1H), 5.56 (m, 1H), 5.30-5.19 (m, 2H), 5.14 (dd, J=10.0, 6.1 Hz, 1H), 4.48 (d, J=4.0 Hz, 1H), 3.66 (m, 1H), 3.50 (m, 1H), 3.40-3.11 (m, 5H), 2.82-2.58 (m, 9H), 2.30 (s, 3H), 2.27 (m, 1H), 2.06 (m, 1H), 1.80 (d, J=6.8 Hz, 3H); HRMS (ESI⁺) calc. for C₄₀H₄₃N₄O₁₀+(M^{m}) 739.2974, found 739.2974.

### Example 13: Synthesis of Compound T18

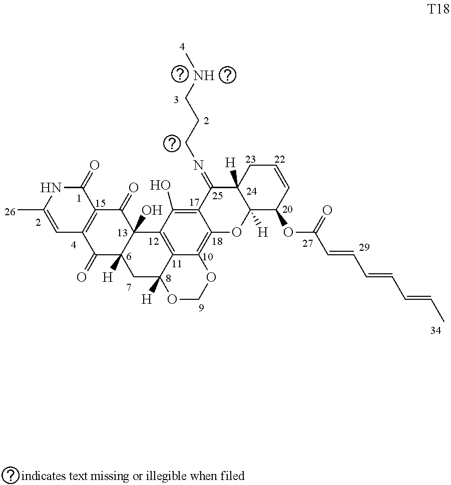 [SMILES: C/C=C/C=C/C=C/C(=O)O[C@@H]1C=CC[C@H]2C(=NCCCNC)c3c(O)c4c5c(c3O[C@H]12)OCO[C@@H]5C[C@@H]1C(=O)c2cc(C)[nH]c(=O)c2C(=O)[C@]41O]

T18. ¹H NMR (400 MHz, CDCl₃:CD₃OD 1:1) δ 7.35 (dd, J=15.2, 11.3 Hz, 1H), 6.53 (dd, J=14.9, 10.6 Hz, 1H), 6.33 (s, 1H), 6.21 (dd, J=14.9, 11.2 Hz, 1H), 6.14 (dd, J=14.9, 11.2 Hz, 1H), 5.94 (dd, J=14.9, 7.1 Hz, 1H), 5.88 (m, 1H), 5.87 (d, J=14.9 Hz, 1H), 5.63 (d, J=10.6 Hz, 1H), 5.55 (m, 1H), 5.27 (d, J=5.7 Hz, 1H), 5.24 (d, J=5.7 Hz, 1H), 5.15 (dd, J=9.9, 6.2 Hz, 1H), 4.44 (d, J=4.0 Hz, 1H), 3.61 (dt, J=13.8, 6.4 Hz, 1H), 3.47 (dt, J=14.3, 6.2 Hz, 1H), 3.34 (m, 1H), 3.00 (m, 2H), 2.78 (s, 6H), 2.76 (s, 1H), 2.63 (m, 1H), 2.48 (s, 1H), 2.28 (s, 3H), 2.21 (m, 1H), 2.07-1.89 (m, 3H), 1.79 (d, J=6.9 Hz, 3H); HRMS (ESI⁺) calc. for C₃₉H₄₂N₃O₁₀+(M^{m}) 712.2865, found 712.2868.

### Example 14: Synthesis of Compound T19

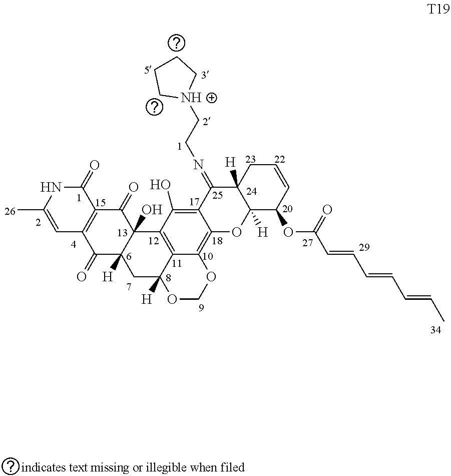 [SMILES: C/C=C/C=C/C=C/C(=O)O[C@@H]1C=CC[C@H]2C(=NCC[NH+]3CCCC3)c3c(O)c4c5c(c3O[C@H]12)OCO[C@@H]5C[C@@H]1C(=O)c2cc(C)[nH]c(=O)c2C(=O)[C@]41O]

T19. ¹H NMR (400 MHz, CDCl₃:CD₃OD 1:1) δ 7.35 (dd, J=15.2, 11.3 Hz, 1H), 6.54 (dd, J=14.8, 10.5 Hz, 1H), 6.36 (s, 1H), 6.22 (dd, J=14.9, 11.2 Hz, 1H), 6.14 (dd, J=14.9, 11.2 Hz, 1H), 5.96 (dt, J=14.8, 7.0 Hz, 1H), 5.92-5.84 (m, 2H), 5.63 (d, J=10.6 Hz, 1H), 5.56 (m, 1H), 5.28 (s, 2H), 5.16 (dd, J=9.8, 6.5 Hz, 1H), 4.47 (d, J=3.9 Hz, 1H), 3.89 (m, 1H), 3.70 (m), 3.39 (dd, J=11.0, 6.8 Hz, 1H), 3.31 (m, 1H); 3.09 (m, 4H), 2.83 (m, 1H); 2.76 (m, 1H), 2.62 (dt, J=13.9, 8.9 Hz, 1H), 2.28 (s, 3H), 2.22 (m, 1H), 1.98 (m, 4H), 1.86 (m, 1H), 1.80 (d, J=6.8 Hz, 3H); HRMS (ESI⁺) calc. for C₄₀H₄₂N₃O₁₀⁺ (M⁺) 724.2865, found 724.2859.

### Example 15: Synthesis of Compound T20

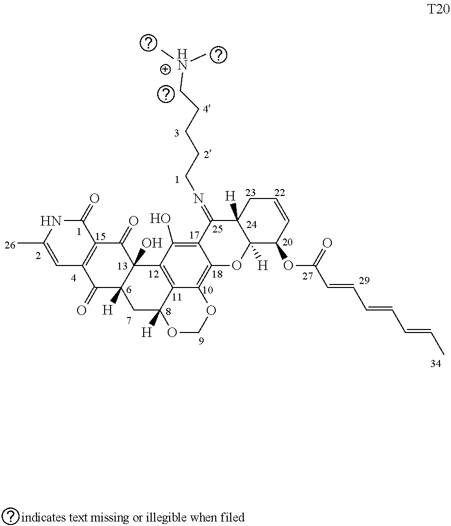

T20. ¹H NMR (400 MHz, CDCl₃: CD₃OD 1:1) δ 7.36 (dd, J=15.2, 11.3 Hz, 1H), 6.54 (dd, J=14.9, 10.7 Hz, 1H), 6.35 (s, 1H), 6.20 (dd, J=14.9, 11.3 Hz, 1H), 6.13 (dd, J=14.9, 11.2 Hz, 1H), 5.95 (dt, J=12.6, 5.7 Hz, 1H), 5.90 (d, J=14.9 Hz, 1H), 5.88 (m, 1H), 5.65 (d, J=10.5 Hz, 1H), 5.56 (m, 1H), 5.26 (d, J=5.7 Hz, 1H), 5.23 (d, J=5.7 Hz, 1H), 5.14 (dd, J=9.8, 6.4 Hz, 1H), 4.48 (m, 1H), 3.59 (m, 1H), 3.44 (m, 1H), 3.36 (m, 1H), 3.19 (m, 3H), 2.82-2.70 (m, 3H), 2.60 (s, 6H), 2.35 (m, 1H), 2.27 (s, 3H), 1.80 (dd, J=6.8, 1.5 Hz, 3H), 1.72-1.60 (m, 4H), 1.50 (m, 2H); HRMS (ESI⁺) calc. for C₄₁H₄₆N₃O₁₀⁺ (M⁺) 740.3178, found 740.3192.

### Example 16: Synthesis of Compound T21

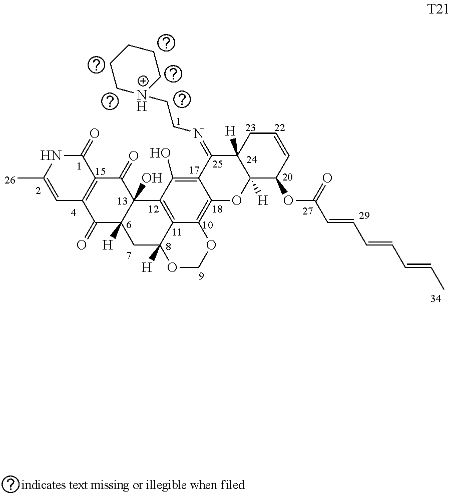 [SMILES: C/C=C/C=C/C=C/C(=O)O[C@@H]1C=CC[C@H]2/C(=N/CC[NH+]3CCCCC3)c3c(O)c4c5c(c3O[C@H]12)OCO[C@@H]5C[C@@H]1C(=O)c2cc(C)[nH]c(=O)c2C(=O)[C@]41O]

T21. ¹H NMR (400 MHz, CDCl₃:CD₃OD 1:1) δ 7.35 (dd, J=15.2, 11.3 Hz, 1H), 6.53 (dd, J=14.8, 10.5 Hz, 1H), 6.37 (s, 1H), 6.21 (dd, J=14.9, 11.2 Hz, 1H), 6.16 (dd, J=14.9, 11.2 Hz, 1H), 5.96 (dt, J=14.8, 7.0 Hz, 1H), 5.92-5.80 (m, 2H), 5.62 (d, J=10.5 Hz, 1H), 5.54 (m, 1H), 5.31-5.23 (m, 2H), 5.18 (dd, J=9.8, 6.4 Hz, 1H), 4.45 (d, J=4.3 Hz, 1H), 3.97 (m, 1H), 3.77 (m, 1H), 3.58 (m, 2H), 3.45-3.36 (m, 4H), 3.34 (m, 1H), 2.93 (m, 3H), 2.80 (m, 1H), 2.63 (m, 1H), 2.29 (s, 3H), 2.20 (m, 1H), 1.91-1.78 (m, 6H), 1.80 (d, J=6.8 Hz, 3H); HRMS (ESI⁺) calc. for C₄₁H₄₄N₃O₁₀⁺ (M⁺) 738.3021, found 738.3024.

### Example 17: Synthesis of Compound T22

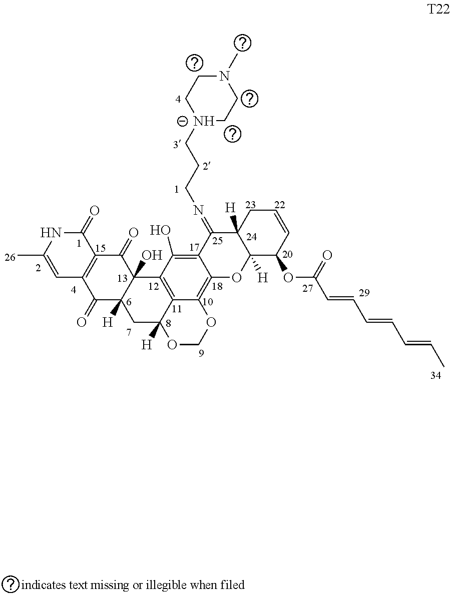

T22. ¹H NMR (400 MHz, CDCl₃:CD₃OD 1.1) δ 7.36 (dd, J=15.2, 11.3 Hz, 1H), 6.54 (dd, J=14.8, 10.6 Hz, 1H), 6.37 (s, 1H), 6.21 (dd, J=14.9, 11.3 Hz, 1H), 6.15 (dd, J=14.9, 11.2 Hz, 1H), 5.99 (dt, J=12.6, 5.7 Hz, 1H), 5.92 (m, 1H), 5.89 (d, J=14.9 Hz, 1H), 5.66 (d, J=10.4 Hz, 1H), 5.58 (s, 1H), 5.28 (d, J=5.7 Hz, 1H), 5.26 (d, J=5.7 Hz, 1H), 5.11 (t, J=8.0 Hz, 1H), 4.49 (m, 1H), 3.64 (m, 1H), 3.52 (m, 1H), 3.39 (m, 1H), 3.30 (m, 1H), 3.05 (m, 4H), 2.78-2.65 (m, 7H), 2.53 (m, 4H), 2.30 (s, 3H), 2.25 (m, 1H), 2.08 (d, J=13.3 Hz), 1.85 (m, 2H), 1.80 (dd, J=6.8, 1.5 Hz, 3H); HRMS (ESI⁺) calc. for C₄₂H₄₇N₄O₁₀⁺ (M⁺) 767.3287, found 767.3293.

### Example 18: Synthesis of Compound T23

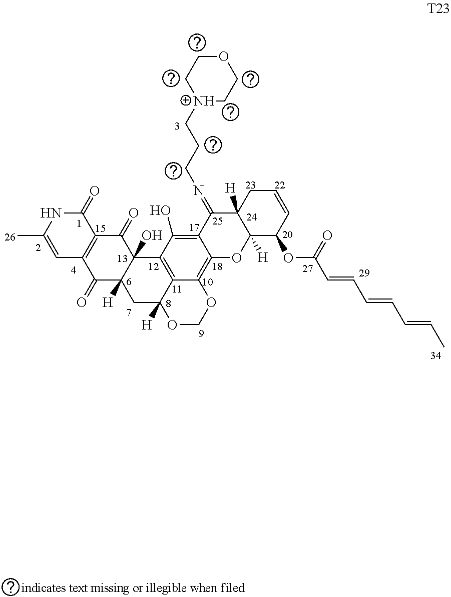 [SMILES: C/C=C/C=C/C=C/C(=O)O[C@@H]1C=CC[C@H]2C(=NCCC[NH+]3CCOCC3)c3c(O)c4c5c(c3O[C@H]12)OCO[C@@H]5C[C@@H]1C(=O)c2cc(C)[nH]c(=O)c2C(=O)[C@]41O]

T23. ¹H NMR (400 MHz, CDCl₃:CD₃OD 1:1) δ 7.36 (dd, J=15.2, 11.3 Hz, 1H), 6.54 (dd, J=14.9, 10.6 Hz, 1H), 6.35 (s, 1H), 6.20 (dd, J=14.9, 11.2 Hz, 1H), 6.13 (dd, J=14.9, 11.2 Hz, 1H), 5.96 (dd, J=14.9, 7.1 Hz, 1H), 5.89 (m, 1H), 5.88 (d, J=14.9 Hz, 1H), 5.65 (d, J=10.5 Hz, 1H), 5.56 (m, 1H), 5.26 (d, J=5.7 Hz, 1H), 5.24 (d, J=5.8 Hz, 1H), 5.14 (dd, J=9.6, 6.6 Hz, 1H), 4.47 (d, J=3.9 Hz, 1H), 3.73 (t, J=4.7 Hz, 4H), 3.59 (m, 1H), 3.48 (m, 1H), 3.39 (dd, J=11.0, 6.9 Hz, 1H), 3.30 (m, 1H), 2.73 (m, 1H), 2.67-2.56 (m, 5H), 2.53 (m, 2H), 2.28 (s, 3H), 2.24 (m, 1H), 2.03 (m, 1H), 1.88 (m, 2H), 1.80 (d, J=6.8 Hz, 3H); HRMS (ESI⁺) calc. for C₄₁H₄₄N₃O₁₁⁺ (M⁺) 754.2970, found 754.2979.

### Example 19: Synthesis of Compound T24

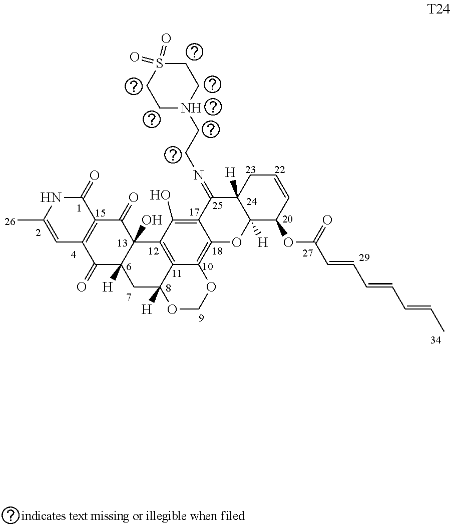 [SMILES: C/C=C/C=C/C=C/C(=O)O[C@@H]1C=CC[C@H]2C(=NCCN3CCS(=O)(=O)CC3)c3c(O)c4c5c(c3O[C@H]12)OCO[C@@H]5C[C@@H]1C(=O)c2cc(C)[nH]c(=O)c2C(=O)[C@]41O]

T24. ¹H NMR (400 MHz, CDCl₃:CD₃OD 1:1) δ 7.36 (dd, J=15.2, 11.3 Hz, 1H), 6.53 (dd, J=14.9, 10.6 Hz, 1H), 6.34 (d, J=8.9 Hz, 1H), 6.19 (dd, J=14.9, 11.3 Hz, 1H), 6.12 (dd, J=14.9, 11.2 Hz, 1H), 5.96 (dd, J=14.9, 7.1 Hz, 1H), 5.91 (m, 1H), 5.88 (d, J=14.9 Hz, 1H), 5.66 (d, J=10.0 Hz, 1H), 5.56 (s, 1H), 5.26 (d, J=5.7 Hz, 1H), 5.24 (d, J=5.7 Hz, 1H), 5.15 (dd, J=9.8, 6.5 Hz, 1H), 4.49 (d, J=3.9 Hz, 1H), 3.65 (m, 1H), 3.46 (dt, J=14.9, 5.4 Hz, 1H), 3.33 (m, 1H), 3.26 (m, 1H), 3.17 (dt, J=12.3, 4.0 Hz, 2H), 3.10-2.94 (m, 6H), 2.87-2.70 (m, 4H), 2.60 (dt, J=13.7, 9.1 Hz, 1H), 2.27 (s, 3H), 2.04 (m, 1H), 1.79 (dd, J=6.8, 1.5 Hz, 3H); HRMS (ESI⁺) calc. for C₄₀H₄₂N₃O₁₂S⁺ (M⁺) 788.2484, found 788.2487.

### Example 20: Synthesis of Compound T25

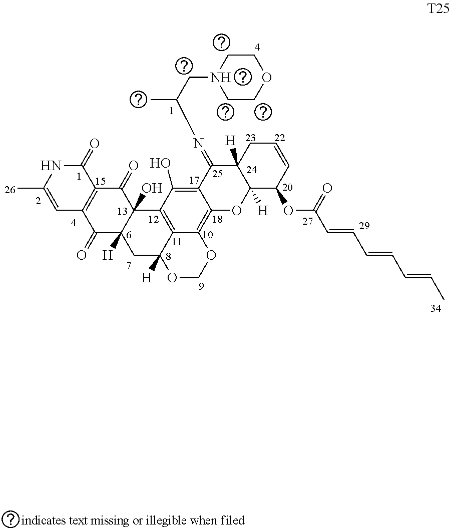 [SMILES: C/C=C/C=C/C=C/C(=O)O[C@@H]1C=CC[C@H]2/C(=N\C(C)CN3CCOCC3)c3c(O)c4c5c(c3O[C@H]12)OCO[C@@H]5C[C@@H]1C(=O)c2cc(C)[nH]c(=O)c2C(=O)[C@]41O]

T25. ¹H NMR (400 MHz, CDCl₃:CD₃OD 1:1) δ 7.37 (dd, J=14.9, 11.2 Hz, 1H), 6.53 (dd, J=14.9, 11.2 Hz, 1H), 6.33 (s, 1H), 6.20 (dd, J=14.9, 11.2 Hz, 1H), 6.13 (dd, J=14.9, 11.2 Hz, 1H), 5.95 (dd, J=14.9, 7.1 Hz, 1H), 5.88 (d, J=14.9 Hz, 1H), 5.87 (m, 1H), 5.65 (d, J=10.4 Hz, 1H), 5.56 (m, 1H), 5.28-5.23 (m, 2H), 5.14 (m, 1H), 4.52 (d, J=4.2 Hz, 1H), 3.96 (m, 1H), 3.66 (m, 1H), 3.51 (m, 5H), 3.44 (t, J=6.1 Hz, 1H), 3.30-3.28 (m, 4H), 2.75 (m, 3H), 2.60 (m, 1H), 2.44 (m, 2H), 2.28 (m, 1H), 2.24 (s, 3H), 1.80 (dd, J=6.8, 1.5 Hz, 3H), 1.17 (d, J=6.3 Hz, 3H); HRMS (ESI⁺) calc. for C₄₁H₄₄N₃O₁₁⁺ (M⁺) 754.2970, found 754.2979.

### Example 21: Synthesis of Compound T26

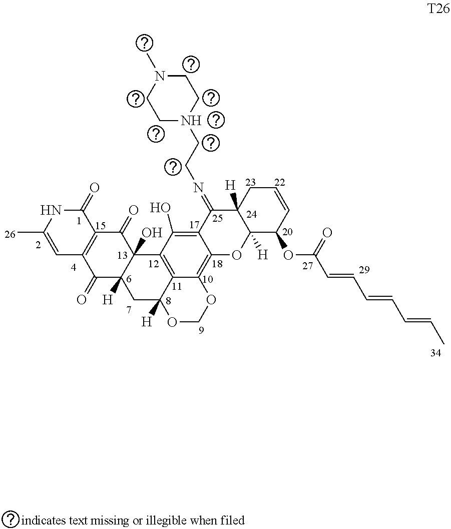 [SMILES: C/C=C/C=C/C=C/C(=O)O[C@@H]1C=CC[C@H]2C(=NCCN3CCN(C)CC3)c3c(O)c4c5c(c3O[C@H]12)OCO[C@@H]5C[C@@H]1C(=O)c2cc(C)[nH]c(=O)c2C(=O)[C@]41O]

T26. ¹H NMR (400 MHz, CDCl₃:CD₃OD 1:1) δ 7.36 (dd, J=15.2, 11.3 Hz, 1H), 6.55 (dd, J=14.9, 10.7 Hz, 1H), 6.33 (s, 1H), 6.21 (dd, J=14.9, 11.3 Hz, 1H), 6.15 (dd, J=14.9, 11.2 Hz, 1H), 5.97 (dt, J=12.6, 5.7 Hz, 1H), 5.92 (m, 1H), 5.89 (d, J=14.9 Hz, 1H), 5.66 (d, J=10.4 Hz, 1H), 5.57 (m, 1H), 5.28-5.22 (m, 2H), 5.14 (dd, J=10.1, 6.1 Hz, 1H), 4.48 (d, J=4.1 Hz, 1H), 3.68 (m, 1H), 3.53 (m, 1H), 3.41 (m, 1H), 3.30 (m, 1H), 3.11-2.95 (m, 7H), 2.86-2.60 (m, 8H), 2.29 (s, 3H), 2.24 (m, 1H), 2.05 (m, 1H), 1.80 (dd, J=6.8, 1.5 Hz, 3H); HRMS (ESI⁺) calc. for C₄₁H₄₅N₄O₁₀⁺ (M⁺) 753.3130, found 753.3137.

### Example 22: Synthesis of Compound T27

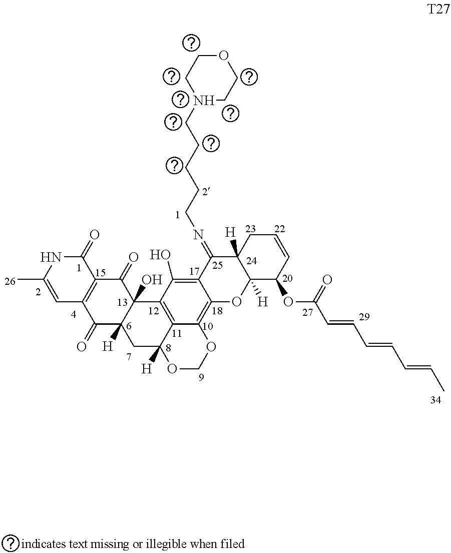 [SMILES: C/C=C/C=C/C=C/C(=O)O[C@@H]1C=CC[C@H]2C(=NCCCCCN3CCOCC3)c3c(O)c4c5c(c3O[C@H]12)OCO[C@@H]5C[C@@H]1C(=O)c2cc(C)[nH]c(=O)c2C(=O)[C@]41O]

T27. ¹H NMR (400 MHz, CDCl₃:CD₃OD 1:1) δ 7.36 (dd, J=15.2, 11.3 Hz, 1H), 6.55 (dd, J=14.8, 10.7 Hz, 1H), 6.38 (s, 1H), 6.21 (dd, J=14.9, 11.2 Hz, 1H), 6.16 (dd, J=14.9, 11.2 Hz, 1H), 5.96 (dd, J=14.9, 7.1 Hz, 1H), 5.90 (m, 1H), 5.89 (d, J=14.9 Hz, 1H), 5.66 (d, J=11.3 Hz, 1H), 5.56 (dq, J=4.1, 2.1 Hz, 1H), 5.27 (d, J=5.7 Hz, 1H), 5.25 (d, J=5.8 Hz, 1H), 5.14 (dd, J=9.8, 6.3 Hz, 1H), 4.40 (m, 1H), 3.94-3.85 (m, 4H), 3.58 (dt, J=13.0, 6.1 Hz, 1H), 3.45 (m, 1H), 3.34 (m, 2H), 3.08-2.90 (m, 4H), 2.89-2.69 (m, 3H), 2.63 (m, 1H), 2.29 (s, 3H), 2.21 (s, 1H), 2.05 (m, 1H), 1.80 (dd, J=6.9, 1.6 Hz, 3H), 1.77-1.61 (m, 4H), 1.48 (m, 1H), 1.36 (m, 1H); HRMS (ESI⁺) calc. for C₄₃H₄₈N₃O₁₁⁺ (M⁺) 782.3283, found 782.3292.

### Example 23: In Vitro Biological Activity

In vitro MIC susceptibility testing. Turbinmicin analogs of the present disclosure were tested for antifungal activity against four Candida isolates, C. albicans K-1, C. tropicalis 98-234, C. auris B 11211, and C. glabrata 4720, and MICs were determined using a broth microdilution method for yeasts. A turbinmicin analog was dissolved in DMSO, serially diluted to 10 concentrations (0.125-64 μg/mL), and tested in a 96-well plate in RPMI medium. Amphotericin B was used as a positive control and the MICs range were 0.25-1.5 μg/mL. Six untreated media controls were included on each plate. The plates were incubated at 33° C. for 24 hours. The MICs were determined as the lowest concentration that inhibited visible growth. The MICs were read at 24. Results are shown in Table 1 for turbinmicin analog activity against various fungi, including drug-resistant fungi.

TABLE 1

| | C. albicans | C. tropicalis | C. auris | C. glabrata |
|---|---|---|---|---|
| | K-1 | 98-234 | B11211 | 4720 |
| Turbinmicin | 0.06 | 0.25 | 0.05 | 0.13 |
| T5 | 2 | 4 | 2 | 4 |
| T6 | >4 | >4 | >4 | >4 |
| T7 | >4 | >4 | >4 | >4 |
| T8 | 0.13 | 0.13 | 0.25 | 0.25 |
| T10 | 2 | 4 | 4 | 1 |
| T11 | 0.06 | 0.06 | 0.06 | 0.13 |
| T12 | 1 | 1 | >4 | 0.5 |
| T13 | >4 | >4 | >4 | >4 |
| T14 | >4 | >4 | >4 | >4 |
| T15 | >4 | >4 | >4 | >4 |
| T16 | 0.38 | 0.5 | 0.06 | 0.25 |
| T17 | 0.5 | 0.5 | 0.25 | 0.5 |
| T18 | 1 | 0.75 | 0.38 | 0.5 |
| T19 | 0.5 | 0.38 | 0.25 | 0.5 |
| T20 | 2 | 4 | 2 | 1 |
| T21 | 0.25 | 0.25 | 0.06 | 0.25 |
| T22 | 1 | 2 | 0.5 | 1 |
| T23 | 1 | 1 | 0.63 | 1 |
| T24 | 0.75 | 1 | 0.28 | 1 |
| T25 | 0.5 | 1 | 0.25 | 0.5 |
| T26 | 0.5 | 0.05 | 0.03 | 0.5 |
| T27 | 1 | 2 | 0.5 | 1 |

Notably, C. auris B 11211, a clinical isolate from India, is highly multidrug-resistant to the three classes of antifungal drugs in current clinical use including fluconazole, micafungin and amphotericin B. C. auris is associated with high morbidity and mortality underscoring the important potential of turbinmicin analogs as an antifungal candidate.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A compound of Formula I: 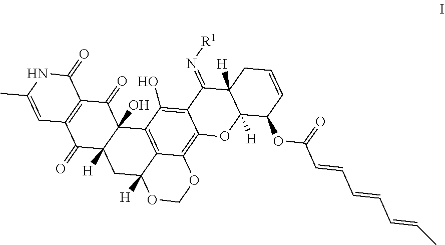
stereoisomers thereof, tautomers thereof, and/or pharmaceutically acceptable salts thereof, wherein
R¹ may be selected from a substituted or unsubstituted alkyl, cycloalkyl, cycloalkylalkyl, alkenyl, cycloalkenyl, alkynyl, aryl, aralkyl, heterocyclyl, heterocyclylalkyl, heteroaryl, or heteroarylalkyl group, or OR²; and
R² may be selected from a substituted or unsubstituted alkyl, alkenyl, alkynyl, aralkyl, or heterocyclylalkyl group.

2. The compound of claim 1, wherein R¹ is selected from a substituted or unsubstituted alkyl, alkenyl, alkynyl, aralkyl, heteroarylalkyl, or heterocyclylalkyl group, or OR², wherein R² is a substituted or unsubstituted alkyl group.

3. The compound of claim 1, wherein R¹ is a substituted or unsubstituted alkyl group.

4. The compound of claim 1, wherein R¹ is a substituted or unsubstituted alkenyl group.

5. The compound of claim 1, wherein R¹ is a substituted or unsubstituted alkynyl group.

6. The compound of claim 1, wherein R¹ is a substituted or unsubstituted aralkyl or heterocyclylalkyl group.

7.-8. (canceled)

9. The compound of claim 1, wherein R¹ and/or R² are independently unsubstituted or substituted with one or more substituents selected from halo, OH, CN, COOH, COOR³, C(O)R³, NO₂, NR⁴R⁵, or C(O)NR⁴R⁵ wherein
R³ at each occurrence is independently H or an unsubstituted alkyl, alkenyl, or aralkyl group; and
R⁴ and R⁵ at each occurrence are independently H or an unsubstituted alkyl, alkenyl, or aralkyl group.

10. The compound of claim 9, wherein R¹ is selected from a C₁₋₆ alkyl, C₂₋₆ alkenyl, or C₂₋₆ alkynyl group, wherein each group is optionally substituted with a substituent selected from OH or NR⁴R⁵.

11. The claim 1, wherein R¹ is selected from aralkyl, heterocyclylalkyl, or heteroarylalkyl groups, each of which is optionally substituted with one or two C₁₋₆ alkyl, aryl, or heteroaryl groups.

12. The compound of claim 11 wherein R¹ is a heterocyclylalkyl group selected from morpholinyl-C₁₋₆ alkyl, piperidinyl-C₁₋₆ alkyl, piperazinyl-C₁₋₆ alkyl, pyrrolidinyl-C₁₋₆ alkyl, thiazolidinyl-C₁₋₆ alkyl, or thiazolidinyl-1,1-dioxide-C₁₋₆ alkyl, thiomorpholinyl-C₁₋₆ alkyl, or thiomorpholinyl-1,1-dioxide-C₁₋₆ alkyl; or R¹ is a heteroarylalkyl selected from pyridinyl-C₁₋₆ alkyl.

13. (canceled)

14. The compound of claim 1, wherein R¹ is unsubstituted piperazinyl-C₁₋₆ alkyl, piperazinyl-C₁₋₆ alkyl substituted with one or two C₁₋₆ alkyl groups, or piperazinyl-C₁₋₆ alkyl substituted with one or two aryl or heteroaryl groups.

15. (canceled)

16. A pharmaceutical composition comprising compound of claim 1 and a pharmaceutically acceptable carrier.

17. The pharmaceutical composition of claim 16 comprising an effective amount of the compound for treating a fungal infection.

18. The pharmaceutical composition of claim 16, wherein the pharmaceutical composition is formulated for oral, parenteral, nasal, or topical administration.

19. The pharmaceutical composition of claim 16, further comprising a second antifungal agent or combination of antifungal agents other than a compound of Formula I.

20. The pharmaceutical composition of claim 19, wherein the second antifungal agent or combination of antifungal agents is selected from the group consisting of azoles, echinocandins, and polyenes.

21. The pharmaceutical composition of claim 19, wherein the second antifungal agent or combination of agents is selected from the group consisting of amphotericin B, flucytosine, fluconazole, voriconazole, posaconazole, isavuconazole, micafungin, cyphomycin, and forazoline.

22. A method of treating a fungal infection comprising administering to a mammal in need thereof an effective amount of a compound of claim 1 or a pharmaceutical composition of claim 16.

23. The method of claim 22, wherein the mammal is human.

24. The method of claim 22, wherein the fungal infection is caused by one or more of Candida, Fusarium, Scedosporium, Rhizopus, Mucor, Apophysomyces, Lichteimia, Cynninghamella or Aspergillus.

25. The method of claim 22, wherein the fungal infection is caused by one or more of Candida albicans, Candida glabrata, Candida auris, Candida tropicalis, Rhizopus delemar, Mucor circinelloides, Apophysomyces elegans, Lichteimia corymbiferea, Aspergillus fumigatus, and drug-resistant strains thereof.

26. The method of claim 22 wherein the effective amount of the compound is 0.01 to 100 mg/kg of body weight in the mammal.

27. (canceled)

28. The method of claim 22, wherein a second antifungal other than the compound of Formula I is administered to the mammal in need thereof simultaneously, sequentially or separately with the compound of Formula I, or the pharmaceutical composition.

29. The method of claim 28, wherein the second antifungal is a selected from the group consisting of amphotericin B, flucytosine, fluconazole, voriconazole, posaconazole, isavuconazole, micafungin, cyphomycin, and forazoline.

30. A method of inhibiting growth of a biofilm comprising one or more of Candida or Aspergillus, the method comprising contacting the biofilm with an effective amount of a compound of claim 1.

31. The method of claim 30, wherein the Candida or Aspergillus is selected from the group consisting of Candida albicans, Candida glabrata, Candida auris, Aspergillus fumigatus, and drug-resistant strains thereof.

32. The compound of claim 1, wherein the compound is selected from the group consisting of 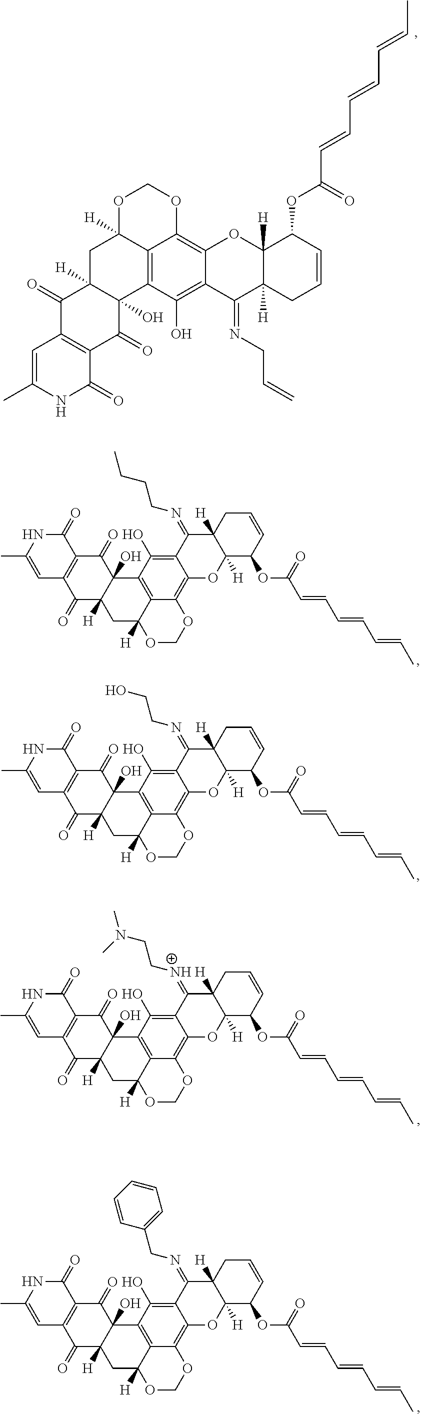 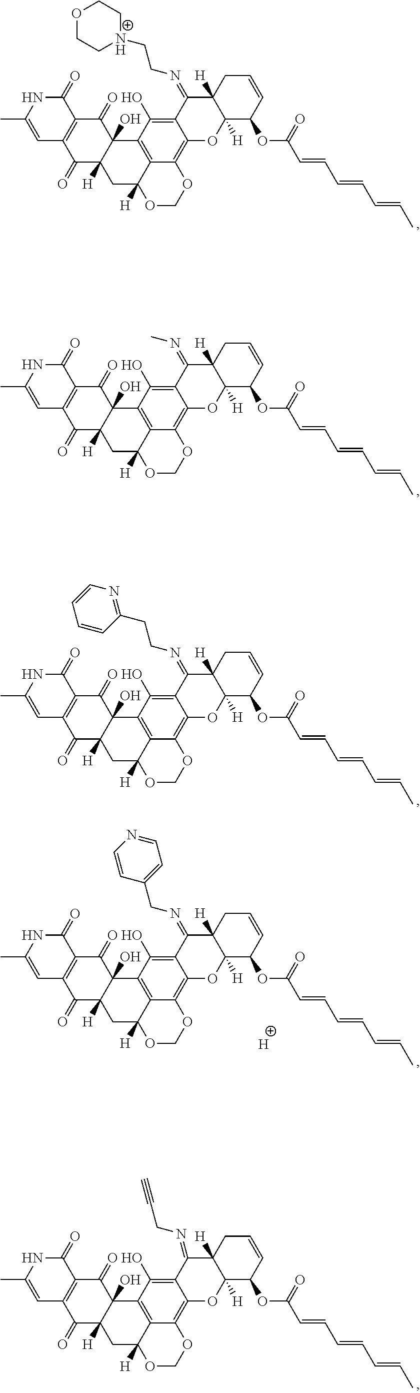 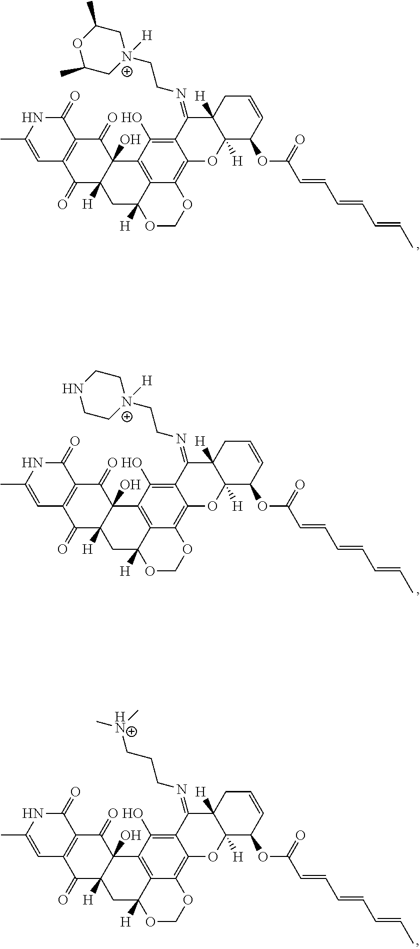 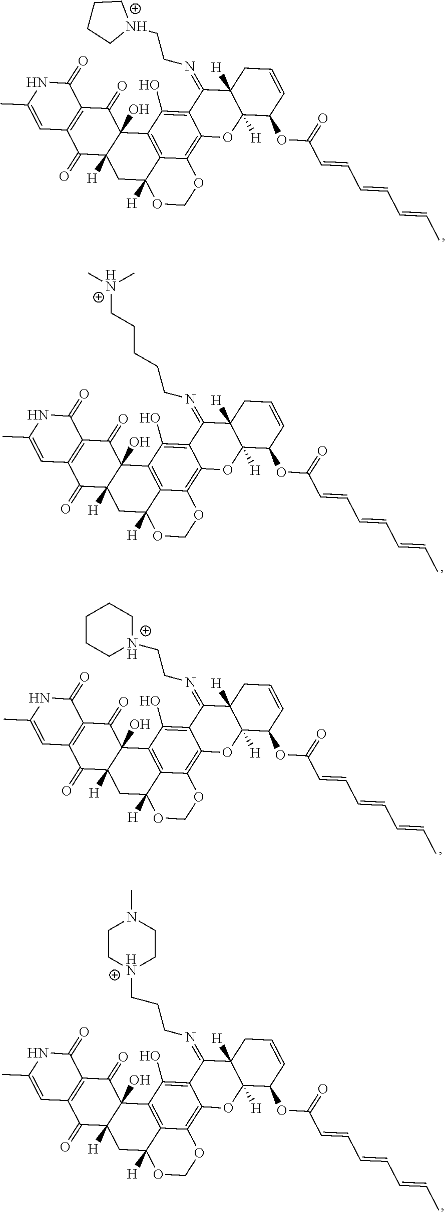 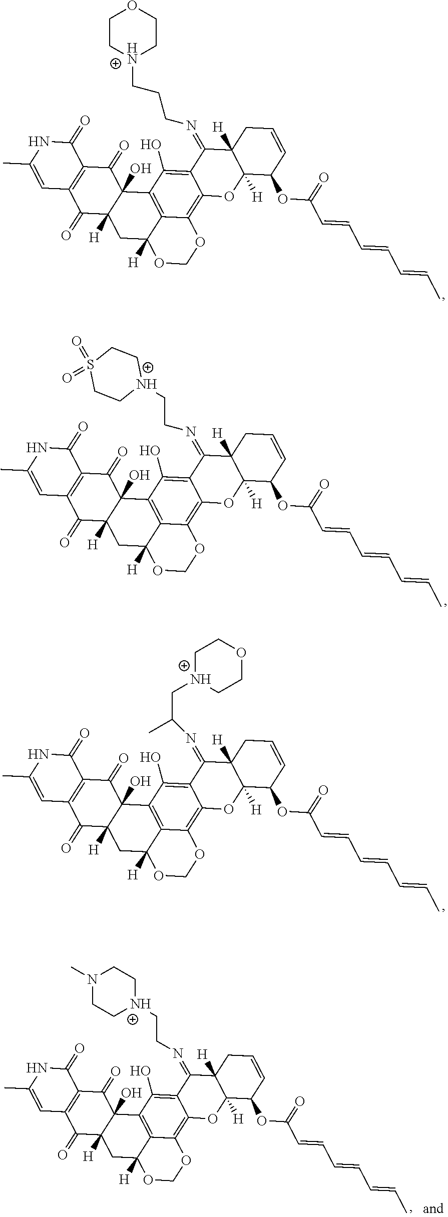 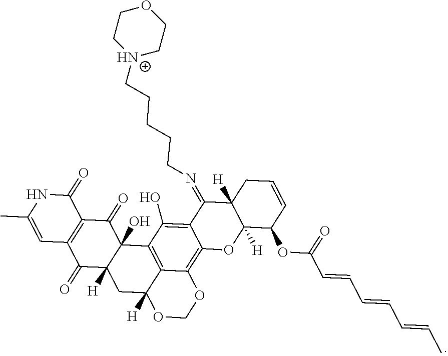 [SMILES: C/C=C/C=C/C=C/C(=O)O[C@@H]1C=CC[C@H]2/C(=N/CCCCC[NH+]3CCOCC3)c3c(O)c4c5c(c3O[C@H]12)OCO[C@@H]5C[C@@H]1C(=O)c2cc(C)[nH]c(=O)c2C(=O)[C@]41O]
